(19) United States
(12) Patent Application Publication
(10) Pub. No.: US 2025/0236957 A1
(43) Pub. Date: Jul. 24, 2025

(54) METHOD FOR PROCESSING FUNCTIONAL MICRO-STRUCTURES ON SURFACE OF METAL BASED ON MASK-ASSISTED JET BIOMACHINING

(71) Applicant: Huaqiao University, Quanzhou (CN)

(72) Inventors: Hui HUANG, Xiamen (CN); Xigui WANG, Xiamen (CN); Jingyu SONG, Xiamen (CN); Weimin LIN, Xiamen (CN); Weibin SHI, Xiamen (CN)

(21) Appl. No.: 19/062,548

(22) Filed: Feb. 25, 2025

Related U.S. Application Data
(63) Continuation of application No. PCT/CN2024/074431, filed on Jan. 29, 2024.

(30) Foreign Application Priority Data
Apr. 12, 2023 (CN) 202310385590.3

(51) Int. Cl. C23F 1/18; C12N 1/20; C12R 1/01; C23F 1/02

(52) U.S. Cl. CPC C23F 1/18; C12N 1/20; C23F 1/02; C12R 2001/01

(57) ABSTRACT

A method for processing micro-structures on a surface of a metal based on mask-assisted jet biomachining, including steps of: (1) culturing microorganisms; (2) preparing the mask; (3) pretreating a surface of a workpiece to be processed; (4) processing the micro-structures; and (5) reoxidizing the microorganisms into a culture liquid for oxidizing.

### RELATED APPLICATIONS

This application is a continuation of International patent application PCT/CN2024/074431, filed on Jan. 29, 2024, which claims priority to Chinese patent application 202310385590.3, filed on Apr. 12, 2023. International patent application PCT/CN2024/074431 and Chinese patent application 202310385590.3 are incorporated herein by reference.

### FIELD OF THE DISCLOSURE

The present disclosure relates to the technical field of processing functional surfaces and specifically relates to a method for processing micro-structures on a surface of a metal based on mask-assisted jet biomachining.

### BACKGROUND OF THE DISCLOSURE

Surface texture refers to processing a pattern on a surface of a material with a preset geometric morphology and size and having a regular arrangement. The surface texture can effectively improve properties of the material, such as wear resistance, optical, biocompatibility, adhesion and detachment, lubrication, heat dissipation, etc. Parts with micro-structure surfaces are widely used in essential components, implants, medical devices, optical elements, and heat exchange devices and have essential application prospects in national strategic industries, such as aerospace, energy, transportation, medical rehabilitation, etc.

The surface texture processing technologies are mainly divided into additive manufacturing, subtractive manufacturing, and molding manufacturing. The additive manufacturing adds materials to the surface of the material in sequence, mainly by coating, sputtering, spraying, deposition, casting, etc., to process micro-protrusions on the surface of the material to improve the performance of the surface of the material. Subtractive manufacturing achieves tiny removal of the surface of the material mainly by mechanical cutting, high-energy beam processing, chemical etching, electrical discharge machining, electrochemical machining, etc., to process tiny grooves, tiny depressions, bionic patterns, etc., on the surface of the material to improve the performance of the surface. The molding manufacturing uses plastic deformation of the material to prepare a large-scale micro-structure under external constraints. In the fabrication of micro-structures ranging from a few to tens of microns thick, the existing processing technologies of the surface texture face problems, such as high equipment costs, high manufacturing costs, difficulty controlling the morphology of micro-nano patterns, and complex processes. Therefore, developing a new processing method to solve the problems mentioned above is necessary.

### BRIEF SUMMARY OF THE DISCLOSURE

The objective of the present disclosure is to solve the deficiencies of the existing techniques and provide a method for processing functional micro-structures on a surface of a metal based on mask-assisted jet biomachining.

In order to achieve the preceding objective, the first technical solution of the present disclosure is as follows. The method for processing the micro-structures on the surface of the metal based on the mask-assisted jet biomachining specifically comprises the following steps.
(1) providing a culture environment for a growth and reproduction of microorganisms, increasing a number and activity of the microorganisms by a scale-up culture, and increasing a concentration of oxidizing ions in a culture liquid synchronously.
(2) designing a pattern of the mask according to a shape and an arrangement of the micro-structures, and preparing the mask.
(3) pretreating a surface of a workpiece to be processed.
(4) adjusting a position between a nozzle of the culture liquid, the mask, and the workpiece to be processed according to a specific morphology of the micro-structures, adjusting process parameters, such as a jet velocity and a jet angle, spraying the culture liquid of the microorganisms having the high concentration of the oxidizing ions onto the surface of the workpiece to be processed through the mask, fully reacting, and removing a bare surface of the workpiece to be processed using the oxidizing ions to form the micro-structures by etching, wherein the jet velocity and the jet angle are key elements for processing an irregular-shaped curved surface and an irregular-shaped micro-structures; and
(5) transporting an oxidized liquid converted from the culture liquid after the etching to microorganisms culturing ware to reoxidize the microorganisms into a culture liquid to continuously recycle a whole processing procedure.

Microorganisms culture is a process of establishing a suitable culture environment in accordance with environmental factors during the growth and metabolism of the microorganisms, such as oxygen, pH value, nutrients, and temperature, providing raw materials to be transformed, and augmenting populations of the microorganisms by a large-scale culture, thereby enhancing activities of the microorganisms and a concentration of oxidizing ions in the culture liquid.

Further, the microorganisms used in the step (1) have a corrosive effect on metals. The microorganisms directly produce ions or participate in ion conversion, and the microorganisms comprise at least one of Thiobacillus ferrooxidans or Thiobacillus thiooxidans.

Further, in a culture process of the step (1), monitoring parameters related to microbial transformation efficiency, such as microbial count and concentration, is necessary during the cultivation process to ensure oxidizing culture liquid. When the activities of the microorganisms are low, the activities of the microorganisms can be gradually increased followed by the volume gradients of different culture liquids.

In a process for processing the micro-structures, the oxidizing liquid fully contacts and reacts with the metal to achieve material removal, and the oxidized liquid generated by the material removal needs to be oxidized to the culture liquid by an action of the microorganisms. Therefore, it is essential to ensure that microbial activity remains optimal during the experiment. The strains are usually stored in a refrigerator, and the activities of the strains are relatively low under conditions of a low temperature, so the activities of the microorganisms need to be increased by gradual culture. Conversion efficiency per unit of time determines the activities of microorganisms. In this method, the activities of microorganisms are characterized by measuring the number of products and several reactants per unit volume after the action of the microorganisms using a color development reaction.

Further, the culture liquid in the step (1) richly comprises the oxidizing ions, and the culture liquid participating in the process for processing the micro-structures can be the culture liquid having the microorganisms or the culture liquid without the microorganisms.

The mask comprises through patterns, and the culture liquid with a preset kinetic energy passes through the mask and fully contacts and reacts with a surface material of the workpiece to be processed.

Further, in the step (2), the shape and the arrangement of the micro-structures are closely related to the enhancement of surface properties, including but not limited to circles, squares, rectangles, or specific geometries.

Further, in the step (2), the scale of the micro-structures are at least one of millimeter, micrometer, or nanometer scales.

Further, in the steps (2) and (4), the mask is made of a material not etched by the culture liquid. Preferably, the mask is plastic, rubber, ceramic, corrosion-resistant metal, etc.

Further, in step (2), appropriate processing methods are selected to fabricate the required masks, including, but not limited to, laser processing, etching, high-energy beam machining, water jet machining, or mechanical machining.

Further, the compositions of the workpiece to be processed include, but are not limited to, pure metallic materials, metal alloy materials, and metal sintered materials. The primary elemental constituents of the workpiece have an oxidation potential lower than that of Fe³⁺, including, but not limited to, iron, cobalt, copper, and tin.

Further, pretreating the surface of the workpiece to be processed in the step (3) comprises and is at least one of removing rusts, degreasing, or polishing, thus eliminating surface residues to obtain a clean and dried surface.

Relative positions of the nozzle, the mask, and the workpiece and processing parameters are appropriately adjusted according to constraints of processing requirements and a processing environment to ensure that a flow field of the culture liquid passing through the mask is less than or equal to a processing scale of the micro-structures on the surface of the workpiece, thereby achieving material removal of a bare section.

Further, after the culture liquid passes through the mask and contacts the surface of the material in the step (4), the oxidizing ions in the culture liquid undergo a displacement reaction with metal monomers or metal oxides on the surface to be processed so that the metal monomers or the metal oxides on the surface are oxidized to metal ions and dissolved in the culture liquid, thus realizing the material removal of the metal. At the same time, the culture liquid involved in the material removal is converted into the oxidized liquid.

Further, in step (4), the cross-section of the nozzle outlet is designed according to the requirements of the micro-structures on the surface of the workpiece, including, but not limited, to circles, squares, rectangles, or other specialized shapes.

Further, in step (4), the jet velocity and the jet angle are adjusted to allow the culture liquid for etching to pass through the unmasked regions and impinge on the surface of the workpiece to be processed. This controls the etching characteristics of the culture liquid for etching, thereby regulating the machining efficiency and precision of the micro-structures on the surface of the workpiece.

Further, in step (4), the pressure of the culture liquid impacting the surface of the workpiece to be processed is adjusted. This allows for the creation of textured surfaces with varying micro-nano-scale structures by leveraging the pressure differences in the culture liquid for etching.

Further, the adjusting the position between the nozzle of the culture liquid and the workpiece to be processed according to the specific morphology of the micro structures on the surface of the workpiece to be processed in the step (4) and the removing the surface of the workpiece to be processed to form the micro-structures on the surface by etching in the step (4) comprises processing the micro-structures on an entity or a part of the surface of the workpiece to be processed.

The oxidized liquid is transported to the microorganisms culturing ware through a pipeline, and an oxidation process from the oxidized liquid to the culture liquid is accelerated using the characteristics of the microorganisms. After the oxidizing characteristics of the culture liquid are recovered, the culture liquid continues to participate in the process of processing the micro-structures, thereby achieving continuous recycling.

Compared with the existing techniques, the present disclosure has the following advantages.
1. The present disclosure utilizes metabolic characteristics of the microorganisms to ensure a high oxidizability of the culture liquid, and a jet technology is introduced to improve a removal efficiency of the material on the surface of the workpiece. This method has characteristics of being green and having low energy consumption;
2. In the process of processing the micro-structures on the surface of the workpiece, the present disclosure adopts a low-energy removal method to reduce damage to a textured surface and improve mechanical properties of the micro-structures on the surface, thus effectively improving the performance of parts and extending the service life of the workpiece;
3. The present disclosure combines a mask technology and a high-precision servo control system to selectively remove the material of the surface of the workpiece, achieving a processing of micro-nano structures of an irregular-shaped workpiece and having strong applicability.

### BRIEF DESCRIPTION OF THE DRAWINGS

FIG. 1 is a diagrammatic view of a method of processing micro-structures on a surface of a metal based on mask-assisted jet biomachining in Embodiment 1, and

FIG. 2 is a diagrammatic view of a method of processing micro-structures of a whole surface of a workpiece by adjusting a posture and a position of the workpiece in Embodiment 1.

List of reference numerals in the drawings: microorganisms reaction vessel 1, culture tank 2, pressure increasing device 3, jet pipeline 4, nozzle 5, mask 6, liquid storage tank 7, copper sample 8, reflux pipeline 9, pump 10, and fixturing platform 11.

### DETAILED DESCRIPTION OF THE EMBODIMENTS

In order to enable the objective, the technical solution, and the advantages of the present disclosure to be clear and definite, the present disclosure will be further described below in conjunction with the accompanying drawings and specific embodiments. However, the protection scope of the present disclosure is not limited to these embodiments. In the description, the same reference numerals always represent identical elements, and similar reference numerals represent similar elements.

In the description of the present disclosure, it should be noted that terms, such as “upper”, “lower”, “front”, “rear”, “left”, “right”, “lateral”, “vertical”, “top”, “bottom”, “inner”, and “outer”, indicate orientations or positional relationships based on orientations or positional relationships shown in the accompanying drawings. These terms are merely used to easily describe the present disclosure and simplify the description of the present disclosure, rather than indicating or implying that a referenced device or element should have a particular orientation or be constructed and operated with a particular orientation, and therefore should not to be understood as a limitation of the present disclosure.

### Embodiment 1

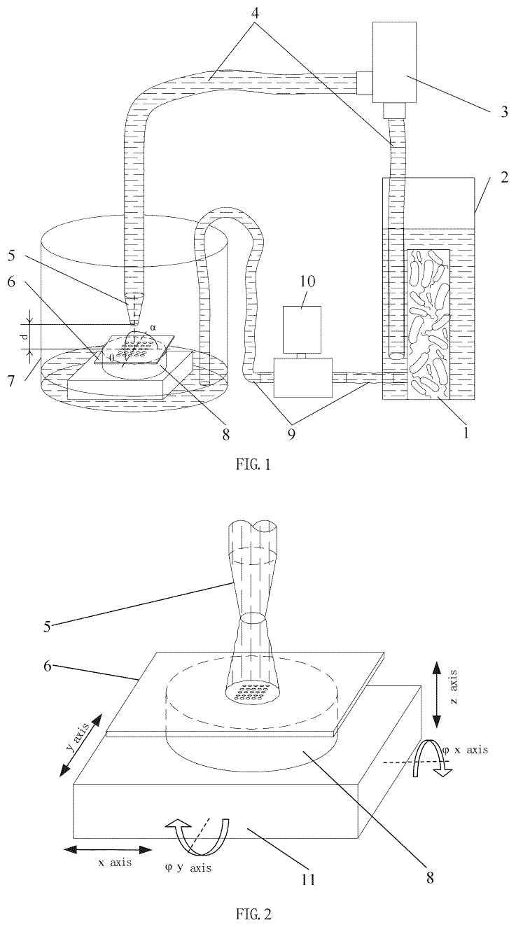

A method for processing micro-structures on a surface of a metal based on mask-assisted jet biomachining comprises the following steps:
1. Microorganism culture: microorganisms used in this embodiment are Thiobacillus ferrooxidans. A direct participation of Thiobacillus ferrooxidans in processing the micro-structures will result in a low processing efficiency due to low activities of the strains of Thiobacillus ferrooxidans at low-temperature storage. In an experiment, a culture liquid with different volumes of 100 mL, 500 mL, 2000 mL, and 10000 mL respectively are used to gradually increase the activities of Thiobacillus ferrooxidans, ensuring Thiobacillus ferrooxidans participating in the experiment have high activities. The culture liquid comprises 3 g/L of ammonium sulfate, 0.5 g/L of magnesium sulfate heptahydrate, 0.1 g/L of dipotassium hydrogen phosphate, 0.01 g/L of potassium nitrate tetracosahydrate, and 24.83 g/L ferrous sulfate heptahydrate. pH of the culture liquid is adjusted to 1.8 using dilute sulfuric acid. The strains of Thiobacillus ferrooxidans are inoculated into the culture liquid, and oxygen is introduced. The culture liquid is cultured in a constant temperature shaker for 20 hours at a temperature of 30° C. and a shaker revolution speed of 180 revolutions per minutes (rpm). Fe²⁺ is converted into Fe³⁺ by microorganisms, and a relative content of Fe²⁺ and Fe³⁺ in the culture liquid is measured to determine an activity of the microorganisms. A solution rich in Fe³⁺ that has been oxidized is used as a culture liquid to participate in processing a surface micro-structure. A concentration of Fe²⁺ in the solution rich in Fe³⁺ is nearly zero, with the majority of the iron ions existing in the form of Fe³⁺. According to color characteristics of Fe²⁺ and Fe³⁺ in different solutions, titration is used to determine whether the conversion is complete.
2. Processing using a mask: as shown in FIGS. 1 and 2, a microscopic pattern of a mask 6 is a matrix of circular holes designed by experience. A 9×9 matrix of the circular holes with a diameter of 50 μm is processed on a 301 stainless steel sheet using a micro-nano laser cutting technology, and the mask tightly fits to a copper sample 8 by mechanical fixturing.
3. Pretreatment of a workpiece to be processed: copper is selected as the workpiece to be processed. The copper sample 8 with a diameter (ϕ) of 15 mm and a thickness (h) of 3 mm is cut from a copper plate and embedded in a resin, and the copper sample 8 is ground and polished by a grinding and polishing machine. The copper sample 8 that is flat and clean is obtained after cleaning and drying.
4. Processing a micro-structure: as shown in FIGS. 1 and 2, a pressure increasing device 3 is used as a power source of a jet in this embodiment to increase a pressure of the culture liquid. The culture liquid in a culture tank 2 is transported to a nozzle 5 through a jet pipeline 4. The culture liquid ejected from an outlet of the nozzle 5 reacts with an exposed surface of the copper sample 8 after passing through the mask 6. A position posture of a fixturing platform 11 can be adjusted to process the micro-structures on a whole surface of the copper sample 8, and a material of a surface of the copper sample 8 is removed. The culture liquid after the reaction is converted into an oxidized liquid and returns to a liquid storage tank 7. A section of the outlet of the nozzle 5 is circular with a diameter of 12 mm, a distance (d) between the outlet of the nozzle 5 and the mask is 5 mm, a flow rate of the culture liquid ejected from the outlet of the nozzle 5 is 0.14 L/s, and an axis of the nozzle 5 is perpendicular to a plane of the mask, that is, an angle α between a nozzle axis and an X-axis of a base plane is 90°, and an angle θ between the nozzle axis and a Y-axis of the base plane is 90°, as shown in FIG. 1. The micro-structures are processed on the surface of the copper sample 8 after the copper sample 8 is etched for 5 minutes.
5. Reoxidizing the culture liquid using metabolic characteristics of the microorganisms: as shown in FIG. 1, the oxidized liquid in the liquid storage tank 7 returns to a microorganisms reaction vessel 1 through a reflux pipeline 9 and a pump
10. The oxidized liquid is reoxidized into the culture liquid using characteristics of the microorganisms and stored in the culture tank 2, so as to achieve a recycling of the culture liquid.

### Embodiment 2

1. Microorganism culture: microorganisms used in this embodiment are Thiobacillus ferrooxidans. A direct participation of Thiobacillus ferrooxidans in processing the micro-structures will result in a low processing efficiency due to low activities of the strains of Thiobacillus ferrooxidans at low-temperature storage. In an experiment, a culture liquid with different volumes of 100 mL, 500 mL, 2000 mL, and 10000 mL respectively are used to gradually increase the activities of Thiobacillus ferrooxidans, ensuring Thiobacillus ferrooxidans participating in the experiment have high activities. The culture liquid comprises 3 g/L of ammonium sulfate, 0.5 g/L of magnesium sulfate heptahydrate, 0.1 g/L of dipotassium hydrogen phosphate, 0.01 g/L of potassium nitrate tetracosahydrate, and 24.83 g/L ferrous sulfate heptahydrate. pH of the culture liquid is adjusted to 1.8 using dilute sulfuric acid. The strains of Thiobacillus ferrooxidans are inoculated into the culture liquid, and oxygen is introduced. The culture liquid is cultured in a constant temperature shaker for 20 hours at a temperature of 30° C. and a shaker revolution speed of 180 revolutions per minutes (rpm). Fe²⁺ is converted into Fe³⁺ by microorganisms, and a relative content of Fe²⁺ and Fe³⁺ in the culture liquid is measured to determine an activity of the microorganisms. A solution rich in Fe³⁺ that has been oxidized is used as a culture liquid to participate in processing a surface micro-structure. A concentration of Fe²⁺ in the solution rich in Fe³⁺ is nearly zero, with the majority of the iron ions existing in the form of Fe³⁺. According to color characteristics of Fe²⁺ and Fe³⁺ in different solutions, titration is used to determine whether the conversion is complete.
2. Processing using a mask: as shown in FIGS. 1 and 2, a microscopic pattern of a mask 6 is a matrix of circular holes designed by experience. A 9×9 matrix of the circular holes with a diameter of 50 μm is processed on a 301 stainless steel sheet using a micro-nano laser cutting technology, and the mask tightly fits to a copper sample 8 by mechanical fixturing.
3. Pretreatment of a workpiece to be processed: copper is selected as the workpiece to be processed. The copper sample 8 with a diameter (ϕ) of 15 mm and a thickness (h) of 3 mm is cut from a copper plate and embedded in a resin, and the copper sample 8 is ground and polished by a grinding and polishing machine. The copper sample 8 that is flat and clean is obtained after cleaning and drying.
4. Processing micro-structures: as shown in FIGS. 1 and 2, a pressure-increasing device 3 is used as a power source of a jet in this embodiment to increase a pressure of the culture liquid. The culture liquid in a culture tank 2 is transported to a nozzle 5 through a jet pipeline 4. The culture liquid ejected from an outlet of the nozzle 5 reacts with an exposed surface of the copper sample 8 after passing through the mask 6. A position posture of a fixturing platform 11 can be adjusted to process the micro-structures on a whole surface of the copper sample 8, and a material of a surface of the copper sample 8 is removed. The culture liquid after the reaction is converted into an oxidized liquid and returns to a liquid storage tank 7. A section of the outlet of the nozzle 5 is circular with a diameter of 12 mm, a distance (d) between the outlet of the nozzle 5 and the mask is 5 mm, a flow rate of the culture liquid ejected from the outlet of the nozzle 5 is 0.17 L/s, and an axis of the nozzle 5 is perpendicular to a plane of the mask, that is, an angle α between a nozzle axis and an X-axis of a base plane is 90°, an angle θ between the nozzle axis and a Y-axis of the base plane is 90°, as shown in FIG. 1. The micro-structures are processed on the surface of the copper sample 8 after the copper sample 8 is etched for 5 minutes.
5. Reoxidizing the culture liquid using metabolic characteristics of the microorganisms: as shown in FIG. 1, the oxidized liquid in the liquid storage tank 7 returns to a microorganisms reaction vessel 1 through a reflux pipeline 9 and a pump
10. The oxidized liquid is reoxidized into the culture liquid using characteristics of the microorganisms and stored in the culture tank 2, so as to achieve a recycling of the culture liquid.

### Embodiment 3

1. Microorganism culture: microorganisms used in this embodiment are Thiobacillus ferrooxidans. A direct participation of Thiobacillus ferrooxidans in processing the micro-structures will result in a low processing efficiency due to low activities of the strains of Thiobacillus ferrooxidans at low-temperature storage. In an experiment, a culture liquid with different volumes of 100 mL, 500 mL, 2000 mL, and 10000 mL respectively are used to gradually increase the activities of Thiobacillus ferrooxidans, ensuring Thiobacillus ferrooxidans participating in the experiment have high activities. The culture liquid comprises 3 g/L of ammonium sulfate, 0.5 g/L of magnesium sulfate heptahydrate, 0.1 g/L of dipotassium hydrogen phosphate, 0.01 g/L of potassium nitrate tetracosahydrate, and 24.83 g/L ferrous sulfate heptahydrate. pH of the culture liquid is adjusted to 1.8 using dilute sulfuric acid. The strains of Thiobacillus ferrooxidans are inoculated into the culture liquid, and oxygen is introduced. The culture liquid is cultured in a constant temperature shaker for 20 hours at a temperature of 30° C. and a shaker revolution speed of 180 revolutions per minutes (rpm). Fe²⁺ is converted into Fe³⁺ by microorganisms, and a relative content of Fe²⁺ and Fe³⁺ in the culture liquid is measured to determine an activity of the microorganisms. A solution rich in Fe³⁺ that has been oxidized is used as a culture liquid to participate in processing a surface micro-structure. A concentration of Fe²⁺ in the solution rich in Fe³⁺ is nearly zero, with the majority of the iron ions existing in the form of Fe³⁺. According to color characteristics of Fe²⁺ and Fe³⁺ in different solutions, titration is used to determine whether the conversion is complete.
2. Processing using a mask: as shown in FIGS. 1 and 2, a microscopic pattern of a mask 6 is a matrix of circular holes designed by experience. A 9×9 matrix of the circular holes with a diameter of 100 μm is processed on a 301 stainless steel sheet using a micro-nano laser cutting technology, and the mask tightly fits to a copper sample 8 by mechanical fixturing.
3. Pretreatment of a workpiece to be processed: copper is selected as the workpiece to be processed. The copper sample 8 with a diameter (ϕ) of 15 mm and a thickness (h) of 3 mm is cut from a copper plate and embedded in a resin, and the copper sample 8 is ground and polished by a grinding and polishing machine. The copper sample 8 that is flat and clean is obtained after cleaning and drying.
4. Processing micro-structures: as shown in FIGS. 1 and 2, a pressure increasing device 3 is used as a power source of a jet in this embodiment to increase a pressure of the culture liquid. The culture liquid in a culture tank 2 is transported to a nozzle 5 through a jet pipeline 4. The culture liquid ejected from an outlet of the nozzle 5 reacts with an exposed surface of the copper sample 8 after passing through the mask 6. A position posture of a fixturing platform 11 can be adjusted to process the micro-structures on a whole surface of the copper sample 8, and a material of a surface of the copper sample 8 is removed. The culture liquid after the reaction is converted into an oxidized liquid and returns to a liquid storage tank 7. A section of the outlet of the nozzle 5 is circular with a diameter of 12 mm, a distance (d) between the outlet of the nozzle 5 and the mask is 5 mm, a flow rate of the culture liquid ejected from the outlet of the nozzle 5 is 0.14 L/s, and an axis of the nozzle 5 is perpendicular to a plane of the mask, that is, an angle α between a nozzle axis and an X-axis of a base plane is 90°, an angle θ between the nozzle axis and a Y-axis of the base plane is 90°, as shown in FIG. 1. The micro-structures are processed on the surface of the copper sample 8 after the copper sample 8 is etched for 5 minutes.
5. Reoxidizing the culture liquid using metabolic characteristics of the microorganisms: as shown in FIG. 1, the oxidized liquid in the liquid storage tank 7 returns to a microorganisms reaction vessel 1 through a reflux pipeline 9 and a pump
10. The oxidized liquid is reoxidized into the culture liquid using characteristics of the microorganisms and stored in the culture tank 2, so as to achieve a recycling of the culture liquid.

The aforementioned description is merely preferred embodiments of the present disclosure, and the present disclosure is not limited thereto. The present disclosure is described in detail in conjunction with the aforementioned embodiments. The technical solution described in the various embodiments can be modified or at least some of the technical features in the various embodiments can be replaced by equivalents by persons of technical skill in the art, and it is intended that any of modifications, equivalents, and improvements will not the depart from the protection scope of the present disclosure provide they are made based on the spirit and the principle of the present disclosure.

What is claimed is:

1. A method for processing micro-structures on a surface of a metal based on mask-assisted jet biomachining, comprising steps of:
(1) providing a culture environment for growth and reproduction of microorganisms, increasing a number and an activity of the microorganisms by a scale-up culture, and increasing a concentration of oxidizing ions in a culture liquid comprising the oxidizing ions and ions to be oxidized;
(2) designing a pattern of a mask according to a shape and an arrangement of the micro-structures, and preparing the mask;
(3) pretreating a surface of a workpiece to be processed; and
(4) adjusting a position between a nozzle of the culture liquid, the mask, and the workpiece to be processed according to a specific morphology of the micro-structures on the surface of the workpiece to be processed, adjusting a jet velocity and a jet angle to spray the culture liquid onto the surface of the workpiece to be processed through the mask, and removing the surface of the workpiece to be processed to form the micro-structures by etching.

2. The method according to claim 1, wherein:
the microorganisms used in the step (1) have a corrosive effect on metals to directly produce ions or participate in ion conversion, and
the microorganisms comprise Thiobacillus ferrooxidans or Thiobacillus thiooxidans.

3. The method according to claim 1, wherein:
in the step (1), the microorganisms participating in processing the micro-structures have activities by monitoring a number or a product concentration of the microorganisms.

4. The method according to claim 1, wherein:
in the step (1), the oxidizing ions are Fe³⁺ which, after oxidizing the metal, are reduced to the ions to be oxidized, and the ions to be oxidized are Fe²⁺.

5. The method according to claim 1, wherein:
in the step (2), the shape and the arrangement of the micro-structures are at least one of circles, squares, rectangles, or specified geometries, and
a scale of the micro-structures is at least one of millimeter, micrometer, or nanometer scales.

6. The method according to claim 1, wherein:
in the steps (2) and (4), the mask is made of a material that is not etched by the culture liquid, and
the pattern of the mask comprises a through pattern.

7. The method according to claim 1, wherein:
in step (2), a method selected for fabricating the mask is laser processing, etching, high-energy beam machining, water jet machining, or mechanical machining.

8. The method according to claim 1, wherein:
primary components of the workpiece to be processed in the steps (3) and (4) are configured to be etched by the culture liquid, and
compositions of the workpiece to be processed comprise at least one of pure metallic materials, metal alloy materials, or metal sintered materials of primary elemental constituents of the compositions having an oxidation potential lower than that of Fe³⁺, and
the primary elemental constituents are at least one of iron, cobalt, copper, or tin.

9. The method according to claim 1, wherein the pretreating the surface of the workpiece to be processed in the step (3) comprises removing rusts, degreasing, and polishing.

10. The method according to claim 1, wherein:
in step (4), a cross-section of an outlet of the nozzle is a circle, a square, a rectangle, or a specialized shape designed according to requirements of the micro-structures on the surface of the workpiece to be processed.

11. The method according to claim 1, wherein:
the adjusting the jet velocity and the jet angle to spray the culture liquid onto the surface of the workpiece to be processed through the mask in the step (4) comprises adjusting an outlet velocity of the nozzle to control a velocity of the culture liquid impacting on the workpiece to be processed after passing through the mask, and adjusting the jet angle to adjust a pressure of the culture liquid impacting on the surface of the workpiece to be processed,
the adjusting the position between the nozzle of the culture liquid and the workpiece to be processed according to the specific morphology of the micro-structures on the surface of the workpiece to be processed in the step (4) comprises controlling a relative position of the nozzle and the workpiece to be processed, and
the removing the surface of the workpiece to be processed to form the micro-structures by etching in the step (4) comprises processing the micro-structures on an entity or a part of the surface of the workpiece to be processed.

12. The method according to claim 1, comprising:
transporting an oxidized liquid converted from the culture liquid in the step (4) after the etching to the step (1) for culturing the microorganisms to reoxidize the microorganisms into the culture liquid for oxidizing.
